(19) United States
(12) Patent Application Publication
(10) Pub. No.: US 2015/0024430 A1
(43) Pub. Date: Jan. 22, 2015

(54) METHOD FOR PRODUCING MILK PROTEIN NANOPARTICLES

(71) Applicant: QMILCH IP GMBH, Hannover (DE)

(72) Inventor: Anke Domaske, Hannover (DE)

(21) Appl. No.: 14/357,247

(22) PCT Filed: Nov. 12, 2012
(86) PCT No.: PCT/EP2012/072424

§ 371 Date: Sep. 10, 2014

(30) Foreign Application Priority Data
Nov. 12, 2011 (DE) 10 2011 118 394.2

(51) Int. Cl. C07K 1/107; C07K 14/76; C07K 14/47; C07K 14/195

(52) U.S. Cl. CPC C07K 1/1077; C07K 14/195; C07K 14/76; C07K 14/4732
USPC 435/68.1; 530/402; 530/410; 530/411; 530/361; 530/366

(57) ABSTRACT

The disclosure relates to milk protein nanoparticles produced according to a polymerization method in which at least one protein, which is obtained from milk and which can be thermally plasticized, is plasticized using a plasticizing agent, such as for example, water or glycerol at temperatures between room temperature and 140° C., subjected to mechanical stress and subsequently retreated, for example, in the top down or bottom up method to form nanoparticles.

### CROSS-REFERENCE TO RELATED APPLICATIONS

This application is a National Stage of International Application No. PCT/EP2012/072424, filed on Nov. 12, 2012, and published in German as WO 2013/068598 A1 on May 16, 2013. This application claims the benefit and priority of German Application No. 10 2011 118 394.2, filed on Nov. 12, 2011. The entire disclosures of the above applications are incorporated herein by reference.

### BACKGROUND

This section provides background information related to the present disclosure which is not necessarily prior art.

1. Technical Field

Methods for producing polymer nano particles are described in literature and known to the man skilled in the art.

2. Discussion

The U.S. Patent Application Publication 20090280148 (Makiko Aimi) describes nano particles on the base of casein, without using surfactants and synthetic polymers, which nano particles have a controllable size, are stable in the acid range and can be produced with another active substance. The casein is however dissolved in a basic, aqueous medium between a pH 8 and a pH 11 in a buffer medium or ethanol. The production of a biopolymer that is basing upon casein and the proof of the nano particles by photometric evaluation has been described, but there is no description with respect to an industrial production. A post-treatment of the corresponding suspension, in order to obtain an economically viable product has neither been described.

The German Patent PCT/EP2007/052320 (BASF SE) “A method for producing polymeric nano particles” comprises semi-synthetic protective colloids, among which also casein, wherein the polymeric product is obtained on the base of polymerizable monomers by using light energy. Herein it is essential with respect to the invention that UV absorbers, a UV treatment and the photo initiator, which is required for this as chemical compound which releases free radicals on exposure to light, are used. Photo initiators are indispensable in the UV treatment since they assure the chemical cross linking process under the UV lamp. It is a fact that, according to EU regulations, already a percentage of 2.5% in the composition requires a labeling with the tree and fish symbol and a disposal as hazardous waste.

In all the other literatures protein based nano particles have also been sufficiently described, but all these literatures use cross linking agents, such as glutaraldehyde or PEG which demonstrably belong to the allergens.

The use of the nano particles which have been mentioned so far shows, in numerous examinations, possibly environmentally harmful and unhealthy aspects of the nano technologies, such as for example the uptake of the particles into the organism via the respiratory passages, the skin and the mouth, even in case of products that are already on the market, such as cosmetics and nutritional supplements.

A question mark has still to be put over concepts concerning the disposal of nano particles. If new disposal regulations are created, it must be considered whether the particles are free or bound to a matrix, whether they are water soluble or not, whether they decompose or congregate. Therefore, possible risks caused by synthetically based polymer nano particles, including the additives and accessory agents for their production, cannot be excluded. It should be aimed at producing nano particles by means of biogen products, if possible, and preferring biodegradable polymers.

### SUMMARY

It is an object of the invention to avoid the above mentioned disadvantages and to preferably produce nano particles from renewable raw materials and preferably without addition of acrylates and fossil raw materials. Besides, it is also an object of the invention to give the nano particles a water and humidity resistance.

Herein, the invention shall in particular reduce the processing time and the use of chemicals, wherein the nano particles shall be preferably, and to the greatest possible extend, produced from renewable and biodegradable raw materials. Simultaneously, the water and energy consumption shall be decreased and the productivity be increased.

This aim is achieved by a method according to the main teachings of the present disclosure.

The present invention is therefore concerned with nano particles which are produced by means of a continuous or discontinuous process of a composition which comprises destructured milk proteins, biodegradable thermoplastic polymers and plasticizers.

Herein, at least one protein obtained from milk or alternatively also a protein produced from bacteria is plasticized together with a plasticizer at temperatures comprised between room temperature and 140° C. under mechanical stress.

The invention is based upon the knowledge that the milk proteins and in particular casein and the derivates thereof can be plasticized and in this manner be polymerized. Preferably it is provided that the plasticizing takes place at temperatures of preferably up to 140° C.

For achieving an even more gentle treatment, the protein is intensely mixed or kneaded with a plasticizer and simultaneously subjected to mechanical stress. Herein, the temperature which is required for the plasticizing is considerably reduced by means of the plasticizer.

The milk protein is preferably casein or lactalbumin or soy protein.

The protein obtained from milk can be produced in situ by precipitation from milk. According to a first procedure, the milk in form of a mixture with lab, other suitable enzymes or acid can be immediately introduced into the process as flocculated mixture or the pressed-off flocculated protein can be used in humid form. According to another optional procedure, a previously separately obtained, if necessary prepared, pure or mixed protein, i.e. a protein fraction from milk, can be used, for example in the form of a dried powder.

The protein fraction can also be produced by ultrafiltration or by using cell cultures. Furthermore, the milk proteins can be modified in other process steps for example by additional salts such as sodium and potassium, such that a casein is produced.

The milk protein used according to the invention can be mixed with other proteins in a proportion of preferably up to 70% by mass with respect to the milk protein. For this, other albumins, such as ovalbumin and vegetable proteins, in particular lupine protein, soy protein or wheat proteins, in particular gluten can be in particular used.

The mixture of solvent and protein is heated up, usually under pressure conditions and shear, in order to accelerate the cross linking process. Chemical and enzymatic agents can also be used, in order to destructurize and to cross link, to oxidize and to derivatize, to etherify, to saponify and to esterify the milk proteins. Usually, the milk proteins are destructurized by dissolving them in water. The milk proteins are completely destructurized, if there are no clots which influence the polymerizing.

In the present invention, a plasticizer can be used in order to destructurize the milk proteins and to enable the milk proteins to flow, i.e. to produce thermoplastic milk proteins. The same plasticizer or other plasticizers can be used in order to increase the melting processability, or two separate plasticizers can be used. The plasticizers can also improve the flexibility of the final products, wherein it is assumed that this is due to the reduction of the glass transition temperature of the composition caused by the plasticizer. The plasticizers are essentially compatible with the polymer constituents of the present invention, such that the plasticizers can effectively modify the properties of the composition. As it is used here, the expression “essentially compatible” means that if the plasticizer is heated up to a higher temperature than the softening and/or melting temperature of the composition, the plasticizer will be able to form an essentially homogenous mixture with milk proteins.

The plasticizer is preferably water which is used in a proportion comprised between 20 and 80% with regard to the weight of the protein, preferably in a proportion comprised between approximately 40 and 50% by mass of the protein content.

Instead of water or mixed with this one, other plasticizers, in particular alcohols, poly alcohols, carbohydrates in aqueous solution and in particular aqueous polysaccharide solutions can be used.

In detail, the following plasticizers and associated proportions are preferred:—hydrogen bridges forming organic compounds without hydroxyl group, for example urea—and derivates,—animal proteins, e.g. gelatin,—vegetable proteins such as for example cotton,—soy beams,—and sunflower proteins,—esters of producing acids which are biodegradable, e.g. citric acid, adipic acid, stearic acid, oleic acid,—hydrocarbon-based acids, e.g. ethylene acrylic acid, ethylene maleic acid, butadiene acrylic acid, butadiene maleic acid, propylene acrylic acid, propylene maleic acid,—sugars, for example maltose, lactose, sucrose, fructose, maltodextrose, glycerin, pentaerythrit and sugar alcohols, e.g. malitol, mannitol, sorbitol, xylitol,—polyols, e.g. hexanetriol, glycols and the like, also mixtures and polymers,—sugar hydrides, e.g. sorbitan,—esters, such as glycerin acetate, (mono, -di, -triacetate) dimethyl and diethylsuccinate and related esters, glycerin propionates, (mono, -di, -tripropionate) butanoates, stereates, phthalate esters. These are non limiting examples of hydroxyl softening agents. Important influencing factors are the affinity to the proteins, the quantity of proteins and the molecular weight. Glycerin and sugar alcohols belong to the most important softening agents. The percentages of the softening agents are for example comprised between 5% and 55%, but they can also be comprised between 2% and 75%. Any desired alcohols, polyols, esters and polyesters can be preferably used in a percentage of up to 30% by weight in the polymer mixture.

The rheological features are of a particular importance for the polymer mixture, in order to achieve a good processing. The solidification under stretch flow is required for forming a stable polymer structure. The melting temperature is mostly in a temperature range comprised between 30° C. and 190° C. Temperatures above these values should be reduced by means of diluents and softening agents.

The biodegradability of the polymers, i.e. their decomposition by living creatures and their enzymes is an important feature of the polymer milk protein nano particles.

Among the biodegradable thermoplastic polymers which are for example suitable for being used in the present invention, are lactic acid polymers, lactide polymers, glycolide polymers, including their homo- and co-polymers and mixtures thereof; aliphatic dibasic dioles/acids; aliphatic polyesteramides, aromatic polyesters, also of modified polyethylene terephthalates and polybutylene terephthalates; polycaprolactones; aliphatic/aromatic copolyesters; poly(3-hydroxyalkanoates), including their copolymers and/or other -valerates, -hexanoates and -alkanoates, polyesters and dialkanoyl polymers, polyamides and copolymers of polyethylene/vinyl alcohol.

These compounds are furthermore suitable as biodegradable thermoplastic polymer of this invention: polyvinyl alcohol and polyvinyl copolymers, aliphatic amide and ester copolymers which are formed by monomers such as for example dialcohols (1,4-butandiol, 1,3-propandiol, 1,6-hexandiol etc.) or ethylene glycol and diethylene glycol, aliphatic polyesteramides, (aliphatic esters are formed with aliphatic amides) or by means of other reactions, such as for example lactic acid with diamines and dicarbonic acid dichlorides, dioles with carbonic acids, caprolacton and caprolactam, or ester prepolymers with diisocyanates, dicarbonic acids, especially succinic acid, oxalic acid and adipic acid and the esters thereof, hydroxycarbonic acids, lactones, amino alcohols (for example ethanolamine, propanolamine), cyclic lactams, aminocarbonic acids (e.g. aminocaproic acid), dicarbonic acids and diamines (e.g. salt mixtures of dicarbonic acids) and mixtures thereof. Polyesters such as for example oligoesters can also be used.

Polybutylene succinate/adipate copolymer; polyalkylene succinates; polypentamethyl succinates; polyhexamethyl succinates; polyheptamethyl succinates; polyoctamethyl succinates; polyalkylene oxalates, such as polyethylene oxalate and polybutylene oxalate polyalkylene succinate copolymers, such as polyethylene succinate/adiapte copolymer and polyalkylene oxalate copolymers, such as polybutylene oxalate/succinate copolymer and polybutylene oxalate/adipate copolymer; polybutylene oxalate/succinate/adipate terpolymers; and mixtures thereof are non limiting examples of aliphatic polyesters of dibasic acids/dioles which are for example produced by polymerization of acids and alcohols or ring-opening reactions and are suitable for producing a polymer.

In the production of biodegradable polymers, aliphatic/aromatic copolyesters can also be used. These copolyesters are formed in a condensation reaction from dicarbonic acids (and derivates) such as malonic, succinic, glutaric, adipic, pimelic, azelaic, sebacic, fumaric, 2,2-dimethyl glutaric, suberic, 1,3-cyclopentane dicarbonic, 1,4-cyclohexane dicarbonic, 1,4-terephtalic, 1,3-terephtalic, 2,6-naphtoeic, 1,5 naphtoeic acid, esters forming derivates and mixtures thereof and dioles, for example ethylene glycol, diethylene glycol, triethylene glycol, tetraethylene glycol, propylene glycol, 1,3-propane diole, 2,2 dimethyl-1,3-propane diole, 1,3-butane diole, 1,4-butane diole, 1,5-pentane diole, 1,6-hexane diole, 2,2,4-trimethyl-1,6-hexane diole, thiodiethanol, 1,3-cyclohexane dimethanol, 1,4-cyclohexane dimethanol, 2,2,4,4-tetramethyl-1,3-cyclobutane diole and combinations thereof. Examples of such aliphatic/aromatic copolyesters include mixtures of poly(tetramethylene glutarate-co-terephthalate), poly(tetramethylene glutarate-co-terephthalate), poly(tetramethylene glutarate-co-terephthalate), poly(tetramethylene glutarate-co-terephthalate), poly(tetramethylene glutarate-co-terephthalate-co-diglocalate), poly(ethyleneglutarate-co-terephthalate), poly(tetramethyleneadipate-co-terephthalate), a mixture having a ratio of 85/15 of poly(tetramethylenesuccinate-co-terephthalate), poly(tetramethylene-co-ethylene-glutarate-co-terephthalate), poly(tetramethylene-co-ethyleneglutarate-co terephthalate). The processability of the protein mass can be modified by other materials, in order to influence the physical and mechanical properties of the protein mass, but also those of the final product. Non limiting examples include thermoplastic polymers, crystallization accelerators or inhibitors, odor masking agents, cross linking agents, emulsifiers, salts, lubricants, surfactants, cyclodextrines, greasing agents, other optical brighteners, antioxidants, processing agents, flame retardants, dye stuffs, pigments, filler materials, proteins and their alkali salts, waxes, adhesive resins and mixtures thereof. These additives are bound to the protein matrix and influence the properties of this one.

Salts can be added to the molten mass. Non limiting examples of salts include sodium chloride, potassium chloride, sodium sulfate, ammonium sulfate and mixtures thereof.

Salts can influence the solubility of the protein in water, but also the mechanical properties. Salts can serve as binding agents between the protein molecules.

Lubricants can, on the other hand, influence the stability of the polymer. These ones can reduce the stickiness of the polymer and decrease the friction coefficient. Polyethylene is a non limiting example.

The physical properties of the polymer mass can be influenced by other proteins; these ones include, without limitation, vegetable proteins such as sunflower protein or animal proteins such as gelatine. Water soluble polysaccharides and water soluble synthetic polymers such as polyacrylic acids can also influence the mechanical properties.

Monoglycerides and diglycerides and phosphatides as well as other animal and vegetable fats can influence and favour the flow characteristics of the biopolymer.

Inorganic filler materials also belong to the optional additives and can be used as processing agents. Possible examples, which do not limit the use, are oxides, silicates, carbonates, lime, clay, limestone and kieselguhr and inorganic salts. Stearate based salts and colophony can be used for modifying the protein mixture.

Amino acids which are constituents of the proteins and peptides can be added to the polymer mass in order to enhance special pleated sheet structures or mechanical properties. Without limitation, glutamic acid, histidine, trytophane etc. are mentioned as examples.

Enzymes, surfactants, acids, serpines as well as phenolic plant molecules are other additives which can contribute as cross linking agents to improve the mechanical properties and the resistance in water and the protease resistance.

Other additives can be desirable in dependence on the respective final use of the intended product. Wet strength is for example a required property of most of the products. Therefore, it is required to add resins comprising a wet-strength as cross linking agents.

Other natural products can also be added as additives. Possible examples of natural polymers are, without limiting the selection, albumins, soy protein, zein protein, chitosan and cellulose, “polylactide” and “PLA”, which can be used in a percentage comprised between 0.1% and 80%.

Apart from natural polymers, other synthetic polymers such as inter alia polyvinyl alcohol as well as polyester or ethers such as polyethylene glycol, aldehydes such as glutaraldehyde and acrylic acids can be used.

These ones also include non-degradable polymers which are used in dependence on the final use of the synthetic material. Thermoplastic synthetic materials which can be used for copolymerization are included, such as—without having a limiting effect—polypropylene, polyethylene, polyamide, polyester and copolymers thereof. Other high molecular polymers are also possible.

Hydrocarbons and polysaccharides as well as amyloses, oligosaccharides and chenodesoxicholic acids can be used as other auxiliary agents and additives.

Salts, carbonic acids, dicarbonic acids and carbonates as well as their anhydrides, salts and esters can also be used as additional cross linking agents. Hydroxides, butylesters as well as aliphatic hydrocarbons present other possibilities to cross link the molecules to each other and to form macromolecules.

The addition of other agents is not excluded. Additives and auxiliary agents, such as lipophile, hydrophobic, hydrophile, hydroscopic additions, glossing agents and crosslinking agents can be especially provided. The additives and auxiliary agents shall altogether not exceed a proportion of maximum approximately 30% by mass with regard to the protein. Vegetable oils, alcohols, fats can be chosen as lipophile additions which slightly hydrophobize the fiber already during the plasticizing operation. Furthermore, waxes and fats can be used which additionally give the fiber stability. Preferred waxes are carnauba wax, beeswax, candelilla wax and other naturally obtained waxes.

After the nano particles have been formed, the nano particles can be further processed or the bound substance can be treated. A hydrophile or hydrophobic surface treatment can be added, in order to adjust the surface energy and the chemical condition of the fabric. Hydrophobic nano particles or the polymer can be for example treated with wetting agents, in order to facilitate the absorption of aqueous liquids. A bound substance can also be treated with a topic solution which contains surfactants, pigments, lubricants, salt, enzymes or other materials, in order to further adjust the surface properties of the MP nano particles or the polymer mass.

For achieving that the milk protein nano particles or the polymer mass thereof meet the stricter requirements with respect to improved properties for a certain purpose, they are, apart from the production methods that have been known and described so far, preferably produced in a bottom up or top down method with the required viscosity. This serves to increase the productivity. The polymer mass is produced by the continuous or discontinuous method which is known to the man skilled in the art and from literature, preferably by mixing or extruding a pre-mixture while adding additives or by preparing the polymer mass by dosing in the basic materials and additives during the mixing or extruding.

The method in which water is used as solvent and plasticizer prevents any difficulties with respect to labour law, toxicology and product approval.

Thanks to the plasticizing operation, the polymer mass corresponds to a polymer in which the materials are transferred into a plastic state by heating them up and are deformed in this manner. Herein, the temperature exceeds the glass transition temperature of the protein such that this one is converted from the amorphous state into the rubber-like plastic state.

After the polymer mass has left for example the extruder, this mass can be immediately processed further, preferably for forming nano particles in the top-down method.

The polymer mass can be further processed to nano particles either immediately after leaving the jet or in at least one later process step.

As a further development of the invention, the polymer mass can also pass through a bath before the hardening, wherein this process is not especially preferred and usually not required. Alternatively, the polymer mass can be subjected to a spraying treatment after having left the jet. Herein, for example smoothing agents, waxes, lipophiles or cross linking agents can be applied to the surface of the polymer mass. In the case of cross linking agents, the following ones are preferred: generally different salt solutions, preferably a calcium chloride solution, a dialdehyde starch solution or an aqueous lactic acid. Alternatively or additionally, the nano particles or the polymer can be subjected to a gas treatment or an ice treatment or a drying and blowing treatment or a ionic treatment or a UV treatment or an enzymatic treatment as well as to a renaturation by means of salts or esterification, etherification, saponification or another cross linking process as well as to a needling and hydro entangling process and to calendaring etc.

The obtained nano particles and the products which are made of these ones can be used for all imaginable purposes. Thus, they can be used for all types of medical biomaterials, in the field of cosmetics, as skin care products, for example also with UV protection, as covering materials, in the food sector, as industrial substances, in medical technology, etc. Best use can also be made of polymers in the fields of textiles and manufacturing of paper.

The nano particles of the present invention which are composed of several constituents can be present in many different configurations. Constituent, such as used here, means, according to definition, the chemical substance or the material. Nano particles can comprise mono component or multiple component configurations. Component, such as used here, is defined as a separate part of the nano particles which is in a spatial relationship with another part of the nano particles. The obtained nano particles can be again applied to a matrix.

The advantages obtained by the invention are inter alia that, in the production of nano particles according to the invention, it becomes possible to reduce the substances which present a health risk and are environmentally harmful during the process and in the nano particles themselves. Besides, the nano particles are biodegradable.

Furthermore, considerable resources of energy, water, time and manpower can be saved, which enhances the environmental protection and improves the economic efficiency. The particularly advantageous properties of the milk protein nano particles are attributed to solidifying structural changes (tertiary structure) during the plasticizing operation.

The nanoscale particles which preferably comprise a diameter of 80-500 nanometers are preferably produced in a top down or bottom up method in order to enable a highest possible productivity. All production methods of the described nano particles, which are known to the man skilled in the art and from literature, can be used without any exception. It is essential with respect to the invention that a homogenously plasticized polymer, preferably a biogen biopolymer, can be produced that is biodegradable. Unfortunately, it has not been possible so far to develop nano particles on this base which are water resistant and sufficiently resistant to proteases, acids and alkalis. Preferably, the use of petroleum-based raw materials and/or solvents, in particular with nano particles which get into contact with food or are even used in medical technical products or in hygiene products or childcare articles, just to mention a few examples, shall be reduced or even excluded.

For nano particles which are preferably produced from renewable raw materials with a proportion of milk protein and are characterized by features such as water resistance, sufficient mechanical properties such as tensile strength and tear resistance, and are elastic, antibacterial and biodegradable, it is furthermore possible to influence the properties of the protein nano particles according to the requirements of the intended purpose by changing the additions of raw materials.

### EXAMPLES

In the following, the invention will be described in detail by means of an exemplary embodiment. The exemplary embodiment only serves to illustrating purposes and shall not limit the invention. On the base of this exemplary embodiment and his know-how, the man skilled in the art can find other possible embodiments by varying the parameters.

### Example 1

Production of a milk protein polymer mass. The extrusion is realized by a twin-screw extruder type 30 E of the company Dr. Collin having a diameter of 30 mm. The nano particles are produced in that the extruded polymer mass is afterwards pulverized to nano particles by high energy ball mills.

The heating is realized by four cylinder heating zones with the following temperature development: 65° C., 74° C., 75° C., 60° C.:

| | temperature | | | | | |
|---|---|---|---|---|---|---|
| | 65 | 74 | 74 | 74 | 75 | 60 |
| function | material | water | plasticizing | outlet zone | head | Jet |
| | supply | supply | zone | | | |
| heating | I | II | II | II | III | IV |
| zone | | | | | | |

The casein powder is supplied via a vibrating conveyor. Water is added by means of a peristaltic pump. The additives are added by means of other dosing devices. The polymer mass is processed to form nano particles by a top down method, for example by a laser removal process. The nano particles can for example have a diameter of 80 mm.

### BRIEF DESCRIPTION OF THE DRAWING

The drawing described herein is for illustrative purposes only of selected embodiments and not all possible implementations, and is not intended to limit the scope of the present disclosure.

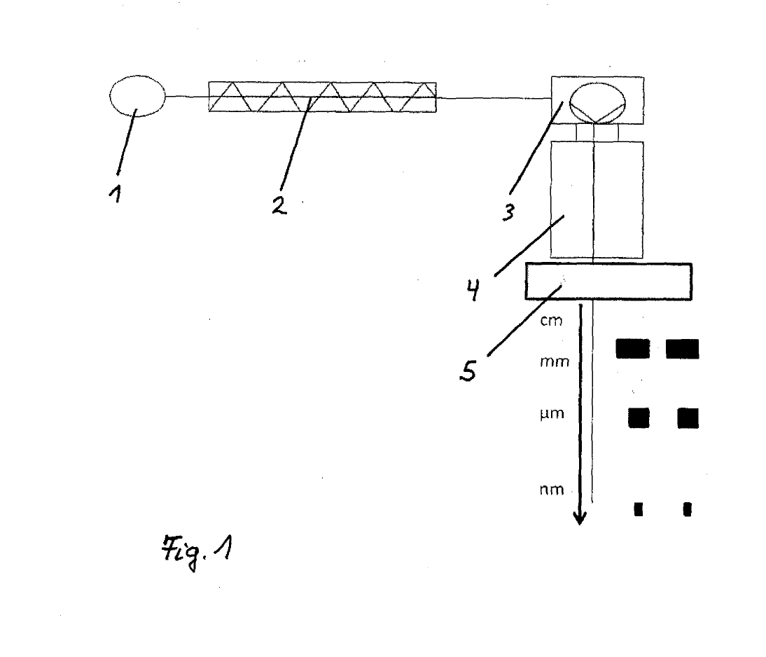

The course of the alternative extrusion process, in which the polymer mass is processed to form nano particles, becomes additionally apparent in FIG. 1.

### DETAILED DESCRIPTION OF THE PREFERRED EMBODIMENTS

Example embodiments will now be described more fully with reference to the accompanying drawing.

The raw materials are dosed into the extruder via a dosing device 1 and the polymer mass is mixed. After having passed through a jet 3 and a device 4 for infrared radiation and blowing, the polymer mass gets into a grinder 5, where it is optionally ground at different grinding degrees.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method for the production of milk protein nano particles composed of at least one homogenous polymer on the base of proteins obtained from milk, which proteins are plasticized by addition of heat and a plasticizer.

2. A method according to claim 1, characterized in that the production is carried out by means of a top down or bottom up method.

3. A method according to claim 1 or 2, characterized in that the nano particles are produced from biogen and renewable raw materials and are biodegradable.

4. A method according to one of the preceding claims, characterized in that the production of the nano particles is a continuous or a discontinuous process.

5. A method according to one of the preceding claims, characterized in that before the nano particles are produced, the polymer mixture is plasticized by means of a continuous or discontinuous process under mechanical stress.

6. A method according to one of the preceding claims, characterized in that the milk protein nano particles are resistant to water, present no health risk and are biodegradable.

7. A method according to one of the preceding claims, characterized in that at least one protein obtained from milk is plasticized together with a plasticizer under mechanical stress.

8. A method according to one of the preceding claims, characterized in that the plasticizing takes place at temperatures of up to 140° C.

9. A method according to one of the preceding claims, characterized in that the protein obtained from milk is either produced in situ by precipitation from milk or is used in form of a protein that has been separately obtained before and, if required, been prepared or in form of a protein fraction.

10. A method according to one of the preceding claims, characterized in that the proteins obtained from milk are obtained from bacteria.

11. A method according to one of the preceding claims, characterized in that the proteins obtained from milk are obtained by gas treatment or filtration.

12. A method according to one of the preceding claims, characterized in that the proteins obtained from milk, in particular casein, lactalbumin or soy protein are obtained from goat's milk, sheep's milk, cow's milk or soy milk.

13. A method according to one of the preceding claims, characterized in that the plasticizer is selected from the group: water, aqueous hydrocarbon solution and in particular aqueous polysaccharides, oligosaccharides, proteins, alcohol, polyalcohol, fats, acids, amino acid, peptides, salts, cations, enzymes or mixtures of these substances as well as their oxidation.

14. A method according to one of the preceding claims, characterized in that other additives and auxiliary agents are added to the base material, optionally by admixing before or during the plasticizing operation.

15. A method according to one of the preceding claims, characterized in that the nano particles or the polymer are dried and post-treated, in that they pass through a bath or are subjected to a spraying treatment or a gas treatment or an ice treatment or a drying and blowing treatment or a ionic treatment or a UV treatment or a needling and hydro entangling process, an infrared treatment or an enzymatic treatment as well as to a renaturation by means of salts or alcohols, esters and ethers, esterification, etherification or saponification or another cross linking process.

16. A method according to one of the preceding claims, characterized in that carbonic acids, dicarbonic acids and carbonates as well as the salts, esters and aliphatic acids thereof are used for the polymer mixture.

17. A method according to one of the preceding claims, characterized in that the polymer mass or the nano particles are destructured, oxidized or derivatized, etherified, esterified or saponified during or after the process by means of chemical or enzymatic substances.

18. A method according to one of the preceding claims, characterized in that amino acids are added to the polymer mixture.

19. A method according to one of the preceding claims, characterized in that the polymer mass is mixed with or post-treated by protease inhibitors, preferably enzymes, surfactants, acids, serpines, phenolic molecules of plants and/or polysaccharides.

20. A method according to one of the preceding claims, characterized in that aliphatic esters and aliphatic amide copolymers, which are preferably biodegradable, are added to the polymer mixture.
